(19) United States
(12) Patent Application Publication
(10) Pub. No.: US 2026/0108262 A1
(43) Pub. Date: Apr. 23, 2026

(54) DIRECTION GUIDE DEVICE OF THROMBUS EXTRACTION CATHETER

(71) Applicants: TAIPEI MEDICAL UNIVERSITY, TAIPEI CITY (TW); ANDIS MEDICAL TECHNOLOGY CO., LTD., New Taipei City (TW)

(72) Inventors: YI-CHUN HUANG, New Taipei City (TW); YUEH-HSUN LU, Taipei City (TW); CHIA-MING WU, New Taipei City (TW)

(21) Appl. No.: 18/918,082

(22) Filed: Oct. 17, 2024

(51) Int. Cl. A61B 17/22; A61B 17/00

(52) U.S. Cl. CPC A61B 17/22; A61B 17/00234; A61B 2017/00238; A61B 2017/00305; A61B 2017/00323; A61B 2017/00367; A61B 2017/22079

(57) ABSTRACT

A direction guide device of a thrombus extraction catheter includes an operation control unit and a traction member connected to the operation control unit. The operation control unit includes a slide block to which the traction member is connected, or alternatively, the slide block is provided with oscillation members connected to first and second traction members, which are arranged side by side with the thrombus extraction catheter and enclosed in an outer enclosure tube. The traction members are positioned and set, at one end thereof, in a front end portion of the outer enclosure tube. The operation control unit can simply operate the slide block to move frontwards/backwards or operate the oscillation members to oscillate leftwards/rightwards to drive the traction members for causing the outer enclosure tube to exhibit curving at various angles to remotely and synchronously curve the thrombus extraction catheter.

### BACKGROUND OF THE INVENTION

### a Technical Field of the Invention

The present invention relates to a direction guide device of a thrombus extraction catheter, and more particularly to a direction guide device, which, through a simple operation, makes a thrombus extraction catheter remotely and synchronously curving to facilitates a medical operation of extracting blood clots.

### b Description of the Prior Art

The formation of blood clots in blood vessels is a dangerous condition in medicine because it may lead to serious consequences such as stroke and myocardial infarction. Therefore, the medical community has been working on developing more effective methods and devices to effectively remove thrombus attached to the inside wall of blood vessels and restore normal blood flow. The known methods for dealing with thrombus are mainly drug therapy and surgical operations, in which the surgical operations use physical thrombus extraction by inserting a thrombus extraction catheter as thick as a pencil core from the artery in a patient’s groin to deeply reach the patient’s thrombus site, under the guidance of X-ray imaging throughout the operation, and generate a strong negative pressure in the blood vessel with Bernoulli’s Law. The thrombus structure is then destroyed through local injection of water, and the scattered thrombus fragments are sucked into the thrombus extraction catheter along with the water flow in the direction of negative pressure. The thrombus fragments do not move to distant locations to cause damages.

The known physical thrombus extraction technique, as shown in FIG. 11, is to place a thrombus extraction catheter 1 into a blood vessel 2 to move along the blood vessel 2 to the site of the thrombus 3. And then, as shown in FIG. 12, the thrombus 3 is sucked into the thrombus extraction catheter 1 in the negative pressure direction to be removed out of the patient’s body. However, since the blood vessels 2 of the human body are not straight but curved, although the thrombus extraction catheter 1 has different sizes for selection, in clinical practice, the thrombus extraction catheter 1 often gets stuck in a curving portion of the blood vessel 2 when traveling in the blood vessel 2 and becomes difficult to pass. This affects the surgical operation of thrombus extraction.

In view of this, the present invention, based on continuous research and experiments, aims to provide a direction guide device of a thrombus extraction catheter, which allows the thrombus extraction catheter to remotely and synchronously curve through a simple operation to facilitate a medical operation of thrombus extraction.

### SUMMARY OF THE INVENTION

The primary objective of the present invention is to provide direction guide device of a thrombus extraction catheter, which allows the thrombus extraction catheter to remotely and synchronously curve through a simple operation during the performance of a surgical operation of thrombus extraction to help the thrombus extraction catheter easily pass through a curved blood vessel, thereby facilitating performance of a medical operation of thrombus extraction.

The aforesaid direction guide device of the thrombus extraction catheter comprises an operation control unit and a traction member connected to the operation control unit. The operation control unit comprises a slide block, and the traction member is connected to the slide block, or alternatively, the slide block is provided with oscillation members, and the oscillation members are connected to a first traction member and a second traction member. The traction members are arranged side by side with the thrombus extraction catheter and are enclosed in an outer enclosure tube. The traction members have one end positioned and set in a front end portion of the outer enclosure tube. As such, the operation control unit can simply operate the slide block to move frontwards/backwards or operate the oscillation members to oscillate leftwards/rightwards to drive the traction members for causing the outer enclosure tube to exhibit curving at various angles thereby achieving the objective of remotely and synchronously curving the thrombus extraction catheter.

In the aforesaid direction guide device of the thrombus extraction catheter, the operation control unit comprises a base, and the base is formed with a slide channel, and the slide block is arranged in the slide channel, such that the slide block is movable along the slide channel.

In the aforesaid direction guide device of the thrombus extraction catheter, the slide block is formed, in a top thereof, with a notch, and the oscillation members each have a central portion that is formed as a circular portion, and the circular portion is arranged in the notch.

In the aforesaid direction guide device of the thrombus extraction catheter, the oscillation members each have two ends that are respectively mounted to a first link bar and a second link bar, and the first traction member and the second traction member are respectively connected, at one end thereof, to the first link bar and a second link bar, while an opposite end extends outwards from a front end of the base to be arranged side by side with the thrombus extraction catheter and enclosed in an outer enclosure tube, so as to have the opposite ends of the first traction member and the second traction member positioned and set in a front end portion of the outer enclosure tube.

### BRIEF DESCRIPTION OF THE DRAWINGS

FIG. 1 is a perspective view of the present invention.

FIG. 2 is an exploded view of the present invention.

FIG. 3 is a cross-sectional view of the present invention.

FIG. 4 is a view showing a state of operation of the present invention.

FIG. 5 is a perspective view showing a second embodiment of the present invention.

FIG. 6 is an exploded view of the second embodiment of the present invention.

FIG. 7 is a top plan view of the second embodiment of the present invention.

FIG. 8 is a cross-sectional view of the second embodiment of the present invention.

FIG. 9 is a view showing a state of operation of the second embodiment of the present invention.

FIG. 10 is a view showing another state of operation of the second embodiment of the present invention.

FIGS. 11 and 12 are schematic views showing an extraction operation performed with a known thrombus extraction catheter.

### DETAILED DESCRIPTION OF THE PREFERRED EMBODIMENTS

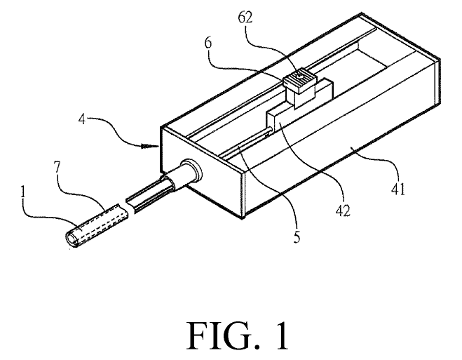

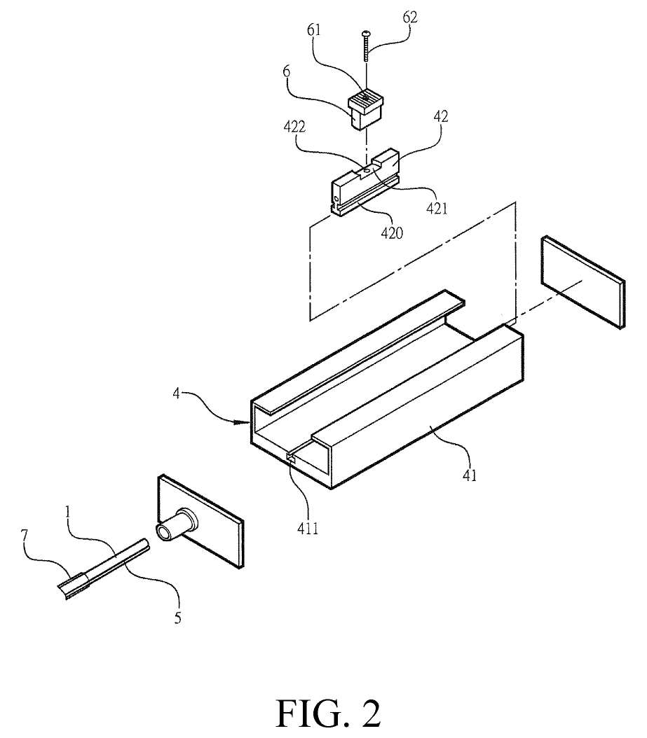

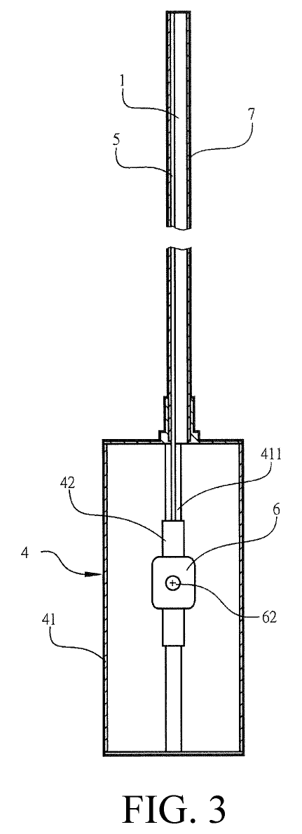

Referring jointly to FIGS. 1 and 2, with additional reference to FIG. 3, which are respectively a perspective view and an exploded view, and a cross-sectional view, of the present invention, as shown in the drawings, the present invention comprises an operation control unit 4 and a first traction member 5 connected to the operation control unit 4. The operation control unit 4 comprises a base 41. A slide channel 411 is formed in the base 41 to allow a slide block 42 to dispose in the slide channel 411 such that the slide block 42 is movable along the slide channel 411. The slide block 42 is formed, in a top thereof, with a notch 421 having two opposite ends that are generally of an arc configuration. A threaded hole 422 is arranged in the notch 421. The slide block 42 has a bottom portion 420 that is formed in an inverted T-shape, so that the inverted T-shape of the bottom portion 420 is fitted in the slide channel 411 and is constrained therein by means of extensions on two opposite sides of a top thereof. A control button 6 having a fastening hole 61 is arranged on the top of slide block 42, and a fastening element 62 extends through the fastening hole 61 to screw into the threaded hole 422 of the slide block 42 to fix the control button 6 on the top of the slide block 42.

One end of the first traction member 5 is connected to the slide block 42, while an opposite end extends outwards through a front end of the base 41 to be arranged side by side with a thrombus extraction catheter 1 and enclosed in an outer enclosure tube 7 to have the opposite end positioned and set in a front end portion of the outer enclosure tube 7. In the instant embodiment, the first traction member 5 is a wire; and the front end portion of the outer enclosure tube 7 comprises a flexible material.

Through the combination of the above-described components, a direction guide device of the thrombus extraction catheter is formed. In a thrombus extraction operation, the control button 6 of the operation control unit 4 can be used to easily operate the slide block 42 to move forwards/backwards along the slide channel 411 to thereby drive the first traction member 5 to move, and the first traction member 5 may drive the outer enclosure tube 7 to curve at various angles in a single direction, achieving the objective of remotely and synchronously curving the thrombus extraction catheter 1. For operations of curving in the opposite direction, it only needs to turn over the operation control unit 4.

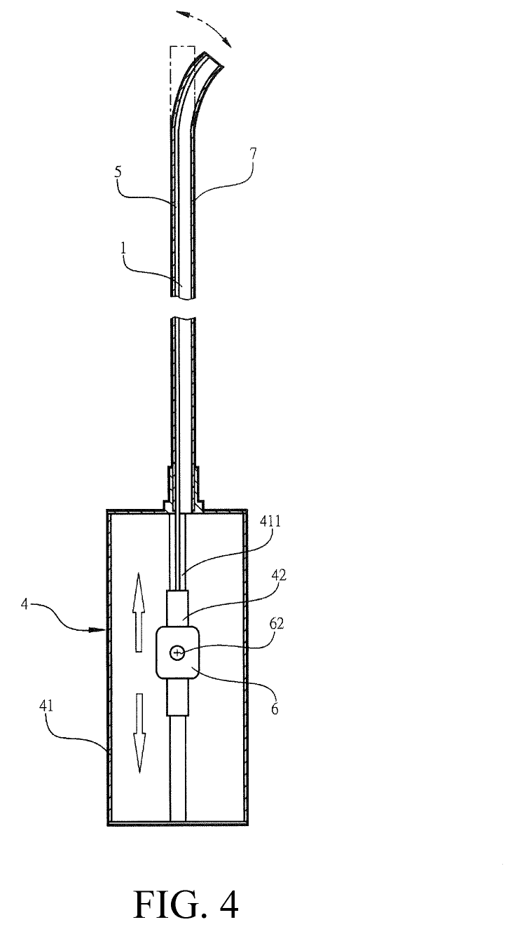

Referring to FIG. 4, is a view showing a state of operation of the present invention is provided. Reference being had to FIGS. 1, 2, 3, and FIGS. 10 and 11, as shown in the drawings, use of the present invention is, similar to the known ways of thrombus extraction operation, to place the outer enclosure tube 7 in which the thrombus extraction catheter 1 and the first traction member 5 arranged side by side therewith are enclosed into a patient’s arterial vessel. When a curve portion of the blood vessel 2 is encountered, the medical professionals may operate the control button 6 to cause the slide block 42 to move frontwards/backwards along the slide channel 411 in order to move the first traction member 5 together therewith to thereby control the outer enclosure tube 7 and the thrombus extraction catheter 1 to curve at different angles. In the instant embodiment, when the control button 6 is moved backwards, the first traction member 5 is pulled backwards and due to the front end portion of the outer enclosure tube 7 being made of a flexible material, the front end portion is caused to curve by the first traction member 5 being pulled backwards. Oppositely, when the control button 6 moves frontwards to restore the set position, the outer enclosure tube 7 and the thrombus extraction catheter 1 resume the state of straight lines. As such, the thrombus extraction catheter 1, when moved in and advancing along the blood vessel 2, may pass through curve portions of the blood vessel 2, avoiding getting stuck in the curve portions and not passing therethrough, so as to make a surgical operation of thrombus extraction carried out in a smoother manner.

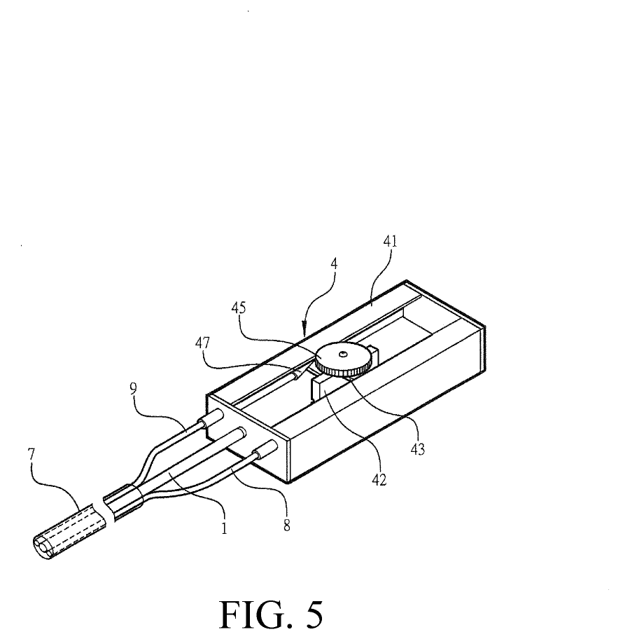

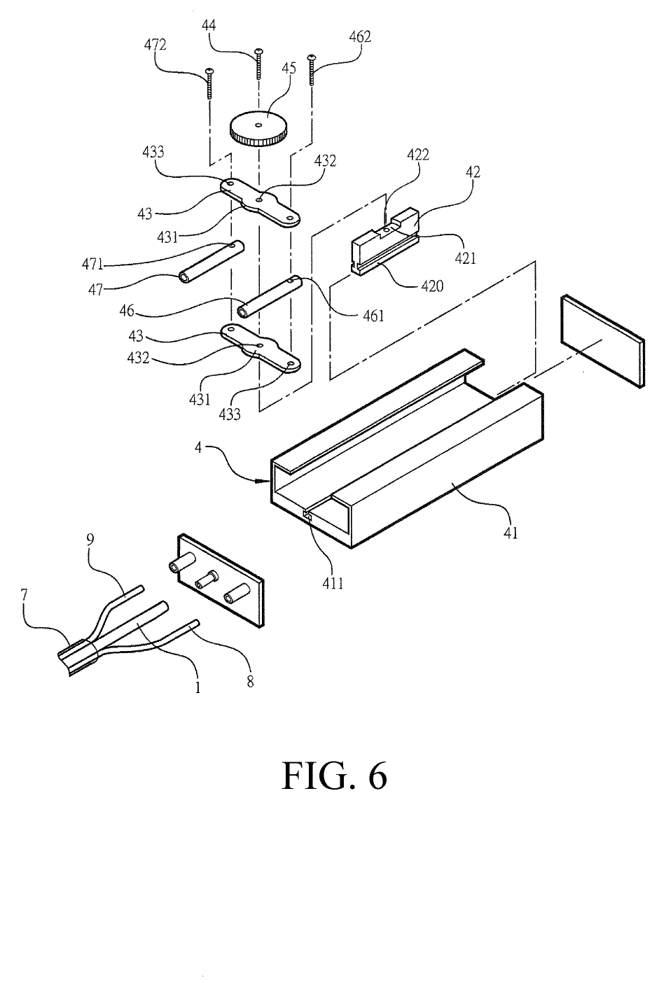

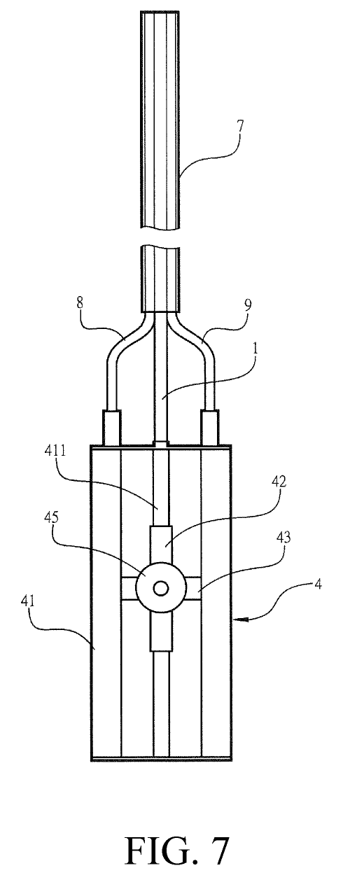

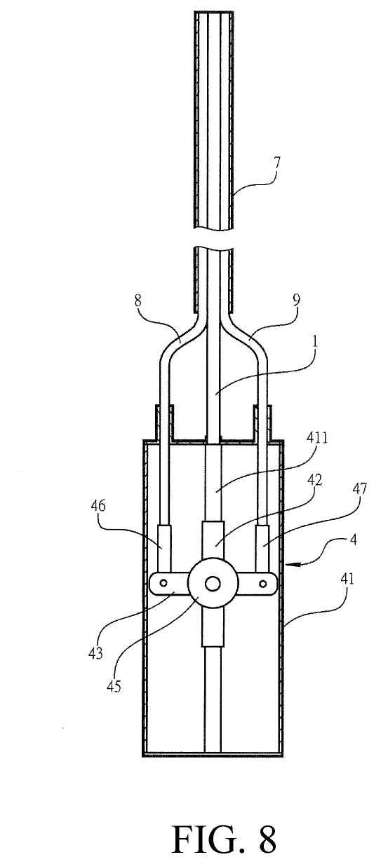

Referring jointly to FIGS. 5 and 6, in combination with FIGS. 7 and 8, which are respectively a perspective view and an exploded view, and a top plan view and a cross-sectional view, of a second embodiment of the present invention, as shown in the drawings, the second embodiment of the present invention comprises an operation control unit 4 and a second traction member 8 and a third traction member 9 connected to the operation control unit 4. The operation control unit 4 comprises a base 41. A slide channel 411 is formed in the base 41 to allow a slide block 42 to dispose in the slide channel 411 such that the slide block 42 is movable along the slide channel 411. The slide block 42 is formed, in a top thereof, with a notch 421 having two opposite ends that are generally of an arc configuration. A threaded hole 422 is arranged in the notch 421. In the instant embodiment, two opposite sides of a top of the slide channel 411 are extended inwardly, and the slide block 42 has a bottom portion 420 that is formed in an inverted T-shape, so that the inverted T-shape of the bottom portion 420 is fitted in the slide channel 411 and is constrained therein by means of the extensions on the two opposite sides of the top thereof.

Two oscillation members 43 are arranged in the notch 421, and a center portion of each of the oscillation members 43 is formed as a circular portion 431. A through hole 432 is formed in the circular portion 431, and a fastening hole 433 is formed in each of two end portions.

The two oscillation members 43 are arranged such that a fastening element 44 penetrates through a rotary knob 45 and then extends through the through holes 432 to screw into the threaded hole 422 of the slide block 42 to have the circular portions 431 exactly located in the notch 421 and the rotary knob 45 located above the oscillation members 43. When the rotary knob 45 is rotated leftwards/rightwards, the two oscillation members 43 are caused to leftwards/rightwards oscillate in the same direction about a center defined by the fastening element 44 and the circular portions 431.

A first link bar 46 and a second link bar 47 are each formed, in one end thereof, with a through hole 461 or a through hole 471, and a fastening element 462 and a fastening element 472 are applied to penetrate through the fastening holes 433 of the two oscillation members 43 and also extend through the through hole 461 and the through hole 471 so as to mount the first link bar 46 and the second link bar 47 between the two oscillation members 43, and also to simultaneously drive, during oscillation of the two oscillation members 43, the first link bar 46 and the second link bar 47 to induce frontward push or backward pull.

The second traction member 8 and the third traction member 9 have ends at one side thereof connected to the first link bar 46 and a second link bar 47 respectively and opposite ends extend outwards through a front end of the base 41 to be each arranged side by side with a thrombus extraction catheter 1 and enclosed in an outer enclosure tube 7 to have the opposite ends positioned and set in a front end portion of the outer enclosure tube 7. In the instant embodiment, the second traction member 8 and the third traction member 9 are each a wire; and the front end portion of the outer enclosure tube 7 comprises a flexible material.

Through the combination of the above-described components, a direction guide device of the thrombus extraction catheter is formed. In a thrombus extraction operation, the rotary knob 45 of the operation control unit 4 can be used to easily operate the oscillation members 43 to oscillate so as to drive the first link bar 46 and the second link bar 47 and the second traction member 8 and the third traction member 9 connected to the first link bar 46 and the second link bar 47, and by means of the second traction member 8 and the third traction member 9 driving the outer enclosure tube 7 to curve at different angles leftwards and rightwards to achieve the objective of remotely and synchronously curving the thrombus extraction catheter 1.

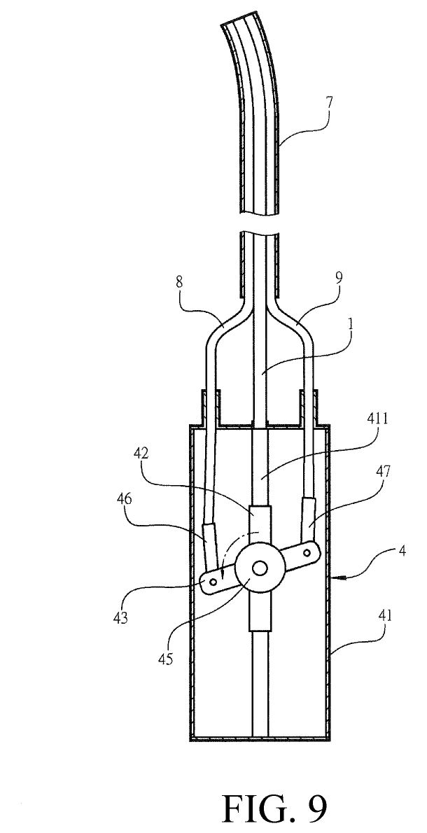

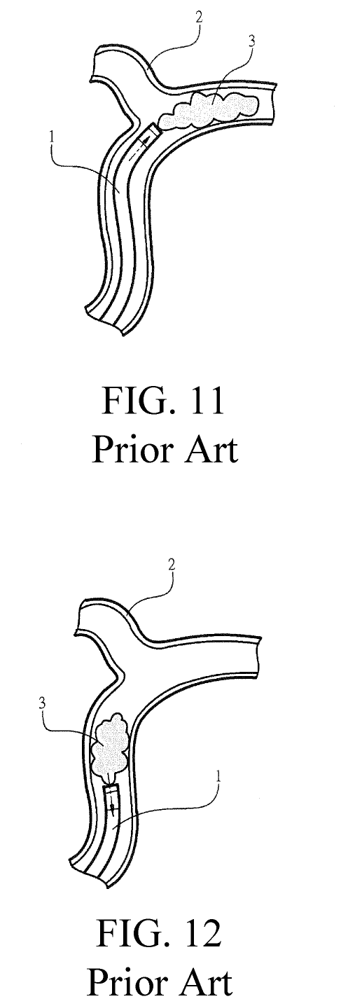

Referring to FIG. 9, a view showing a state of operation of the second embodiment of the present invention is provided. Reference being had to FIGS. 11 and 12, as shown in the drawings, use of the present invention is, similar to the known ways of thrombus extraction operation, to place the outer enclosure tube 7 in which the thrombus extraction catheter 1 and the second traction member 8 and the third traction member 9 arranged side by side therewith are enclosed into a patient’s arterial vessel. When a curve portion of the blood vessel 2 is encountered, the medical professionals may cause the slide block 42 to move along the slide channel 411 to properly adjust the position of the second traction member 8 and the third traction member 9 and may rotate the rotary knob 45 to control the outer enclosure tube 7 to exhibit curving of different angles to thereby control the outer enclosure tube 7 and the thrombus extraction catheter 1 to exhibit curving of different angles. In the instant embodiment, when the rotary knob 45 is rotated leftwards, the first link bar 46 is pulled backwards, while the second link bar 47 is pushed frontwards, making the second traction member 8 pulled backwards and the third traction member 9 pushed frontwards. Due to the front end portion of the outer enclosure tube 7 being made of a flexible material, the front end portion is caused to curve leftwards by means of the second traction member 8 being pulled backwards. As such, the thrombus extraction catheter 1, when moved in and advancing along the blood vessel 2, may pass through curve portions of the blood vessel 2, avoiding getting stuck in the curve portions and not passing therethrough, so as to make a surgical operation of thrombus extraction carried out in a smoother manner.

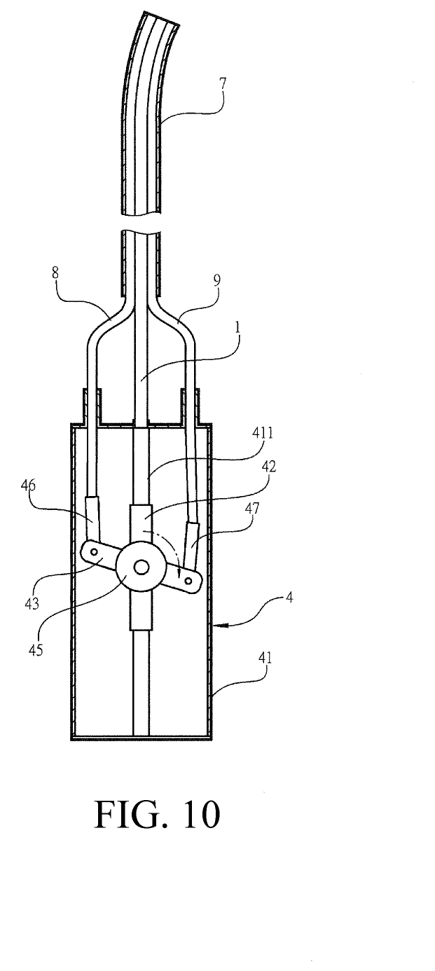

Referring to FIG. 10, a view showing another state of operation of the second embodiment of the present invention is provided. Reference being had to FIGS. 11 and 12, as shown in the drawings, the present invention is operated such that when the rotary knob 45 is rotated rightwards, the second link bar 47 is pulled backwards, while the first link bar 46 is pushed frontwards, making the third traction member 9 pulled backwards and the second traction member 8 pushed frontwards. Due to the front end portion of the outer enclosure tube 7 being made of a flexible material, the front end portion is caused to curve rightwards by means of the third traction member 9 being pulled backwards. As such, the thrombus extraction catheter 1, when moved in and advancing along the blood vessel 2, may pass through curve portions of the blood vessel 2, avoiding getting stuck in the curve portions and not passing therethrough, so as to make a surgical operation of thrombus extraction carried out in a smoother manner.

The embodiments presented above are provided as the preferred ways of implementing the present invention, not intending to limit the scope of the present invention. Minor modifications and variations, which do not depart from the essence of the present invention, fall within the spirit scope of the present invention.

In summary, the present invention uses a base that is provided with a slide channel and a slide block for collaborative connection with the slide block, and a traction member arranged side by side with a thrombus extraction catheter in an enclosed form to constitute a direction guide device of the thrombus extraction catheter. When the thrombus extraction catheter is used in a thrombus extraction operation, a simple operation may be implemented to remotely and synchronously curve the thrombus extraction catheter, allowing the thrombus extraction catheter to easily pass through a curve of a blood vessel, so as to facilitate the performance of a medical operation of thrombus extraction.

I claim:

1. A direction guide device of a thrombus extraction catheter, comprising an operation control unit and a first traction member connected to the operation control unit, wherein the operation control unit comprises a slide block; the first traction member is arranged side by side with the thrombus extraction catheter and is enclosed by an outer enclosure tube; one end of the first traction member is connected to the slide block, while an opposite end is positioned and set in a front end portion of the outer enclosure tube; and as such, the operation control unit is operable to cause the slide block to move frontwards/backwards to drive the first traction member to cause the outer enclosure tube to curve at various angles to thereby achieve an objective of remotely and synchronously curving the thrombus extraction catheter.

2. The direction guide device of the thrombus extraction catheter according to claim 1, wherein the operation control unit comprises a base, and the base is formed with a slide channel, and the slide block is arranged in the slide channel, such that the slide block is movable along the slide channel to drive the first traction member to cause the outer enclosure tube to curve at various angles.

3. A direction guide device of a thrombus extraction catheter, comprising an operation control unit and a second traction member and a third traction member connected to the operation control unit, wherein the operation control unit comprises a slide block and oscillation members pivotally connected to the slide block; the second traction member and the third traction member are arranged side by side with the thrombus extraction catheter and are enclosed by an outer enclosure tube; the second traction member and the third traction member each have an end connected to the oscillation members and an opposite end positioned and set in a front end portion of the outer enclosure tube; and as such, the operation control unit is operable to cause the oscillation members to oscillate leftwards/rightwards to drive the second traction member and the third traction member to cause the outer enclosure tube to curve at various angles to thereby achieve an objective of remotely and synchronously curving the thrombus extraction catheter.

4. The direction guide device of the thrombus extraction catheter according to claim 3, wherein the operation control unit comprises a base, and the base is formed with a slide channel, and the slide block is arranged in the slide channel.

5. The direction guide device of the thrombus extraction catheter according to claim 3, wherein the slide block is formed, in a top thereof, with a notch, and the oscillation members each have a central portion that is formed as a circular portion, wherein the circular portion is pivotally connected in the notch to allow the oscillation members to oscillate leftwards/rightwards about a center defined by the circular portion, so as to drive the second traction member and the third traction member to cause the outer enclosure tube to curve at various angles.

6. The direction guide device of the thrombus extraction catheter according to claim 3, wherein the oscillation members are fastened to a rotary knob, and rotation of the rotary knob causes the oscillation members to oscillate leftwards/rightwards.

7. The direction guide device of the thrombus extraction catheter according to claim 3, wherein the oscillation members each have two ends that are respectively mounted to a first link bar and a second link bar, and the second traction member and the third traction member are respectively connected, at ends thereof, to the first link bar and the second link bar.
